(19) United States
(12) Patent Application Publication
(10) Pub. No.: US 2005/0065414 A1
(43) Pub. Date: Mar. 24, 2005

(54) Pulse oximeter system

(76) Inventors: Robert V. Allen, Nashville, TN (US); Benjamin A. Schnitz, Brentwood, TN (US)

Correspondence Address: MYERS BIGEL SIBLEY & SAJOVEC, PO BOX 37428, RALEIGH, NC, 27627, US

(21) Appl. No.: 10/900,587

(22) Filed: Jul. 23, 2004

Related U.S. Application Data
(60) Provisional application No. 60/489,609, filed on Jul. 24, 2003.

(51) Int. Cl. A61B 5/00

(52) U.S. Cl. 600/310; 128/903

(57) ABSTRACT

A monitoring device for a laboratory animal may include an inductive power receiver, a sensor, and a data transmitter. The inductive power receiver may be configured to generate electrical power responsive to an electromagnetic field, and the sensor may be configured to generate an electrical signal responsive to an aspect of the laboratory animal being monitored. The data transmitter may be configured to wirelessly transmit data responsive to the electrical signal generated by the sensor and responsive to the electrical power generated by the inductive power receiver.

### RELATED APPLICATIONS

The present application claims the benefit of priority from U.S. Provisional Application No. 60/489,609 filed Jul. 24, 2004. The disclosure of U.S. Provisional Application No. 60/489,609 is hereby incorporated herein in its entirety by reference.

### BACKGROUND

Pulse oximetry is a method of monitoring the percentage of haemoglobin (Hb) which is saturated with oxygen. Pulse oximeters are discussed, for example, in U.S. Pat. No. 6,763,256, U.S. Pat. No. 6,760,609, U.S. Pat. No. 6,748,253, and U.S. Pat. No. 6,731,962. The disclosures of each of these patents is hereby incorporated herein in their entirety by reference. Conventional pulse oximeters, however, may be difficult to use on non-human subjects.

### SUMMARY

According to embodiments of the present invention, a monitoring device for a laboratory animal may include an inductive power receiver, a sensor, and a data transmitter. The inductive power receiver may be configured to generate electrical power responsive to an electromagnetic field, and the sensor may be configured to generate an electrical signal responsive to an aspect of the laboratory animal being monitored. The data transmitter may be configured to wirelessly transmit data responsive to the electrical signal generated by the sensor and responsive to the electrical power generated by the inductive power receiver.

### BRIEF DESCRIPTION OF THE DRAWINGS

FIG. 1 is a photograph of a pulse oximeter cape and cuff on a mouse according to embodiments of the present invention.

FIG. 2 is a block diagram of functional elements of a cape and a cuff according to embodiments of the present invention.

FIG. 3 is a block diagram of functional elements of a receiver base according to embodiments of the present invention.

FIG. 4 is a diagram illustrating operations of pulse oximeters according to embodiments of the present invention.

### DETAILED DESCRIPTION

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

As will be appreciated by those of skill in the art, the present invention may be embodied as methods or devices. Accordingly, the present invention may take the form of a hardware embodiment, a software embodiment or an embodiment combining software and hardware aspects. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. As used herein, the term “and/or” includes any and all combinations of one or more of the associated listed items.

It will also be understood that although the terms first, second, etc. are used herein to describe various elements, these elements should not be limited by these terms. These terms are only used to distinguish one element or embodiment from another element or embodiment. Thus, a first element or embodiment could be termed a second element or embodiment, and similarly, a second element or embodiment may be termed a first element or embodiment without departing from the teachings of the present invention.

NovaMouse™ is a family of physiological and diagnostic research devices configured to be attached to and/or implanted in small laboratory animals.

According to particular embodiments of the present invention, the NovaMouse™ pulse oximeter system measures the heart rate and/or hemoglobin saturation level in the arterial blood of the mouse's tail. The NovaMouse™ pulse oximeter system may include two components, the NovaMouse™ flexible cape and the NovaMouse™ receiver. The NovaMouse™ flexible cape is an attachable device for mice which wirelessly relays heart rate and blood oxygen saturation data to a receiver located in the NovaMouse™ receiver base. The NovaMouse™ receiver base may also inductively power the NovaMouse™ flexible cape.

The NovaMouse™ Flexible Cape

The NovaMouse™ flexible cape may include the pulse oximeter and wireless transmitter electronics. Data may be transmitted to the NovaMouse™ receiver base wirelessly—eliminating tethers—and power may be provided from the receiver base to the cape through an inductive power coupling system.

Placement

The pulse oximeter cuff may be located on the proximal end of the mouse's tail where a central artery and two veins are found. The hairless tail provides an ideal location for accurate measurement of pulse and/or oxygen saturation. Moreover, placement at the base of the mouse's tail may inhibit the mouse's ability to detach or damage the cuff by gnawing.

Attachment

The pulse oximeter cuff can be easily attached by sliding it up the mouse's tail until a snug fit is achieved. The NovaMouse™ flexible cape may be made available in different forms. A first has an elastic strap for attachment to the abdominal region to be used for short-term studies. For long-term studies, sutures can be used to more securely attach the NovaMouse™ flexible cape. According to yet another alternative, the cape may be sufficiently resilient to provide a friction fit without requiring a strap or sutures.

Material

The NovaMouse™ flexible cape may be constructed on a flexible polyimide substrate. This lightweight design allows the NovaMouse™ flexible cape to conform to mice.

The NovaMouse™ Receiver Base

The NovaMouse™ receiver base may include wireless communication hardware and/or software, an inductive power coupling system and a data connection such as a universal serial bus (USB) connection for coupling with a computing device such as a personal computer (PC).

Placement

The NovaMouse receiver base may be placed under a standard 7″×11″ laboratory mouse cage. The NovaMouse receiver base may be designed to hold the standard mouse cage securely in place with raised edges along the top of the receiver base. Moreover, the receiver base may be integrated with a laboratory cage.

Power

The NovaMouse™ receiver base may wirelessly power the NovaMouse™ flexible cape via an inductive power coupling system. Accordingly, a wired coupling is not required to power electronics on the cape.

Wireless Communication

The NovaMouse™ receiver base may include the wireless communication hardware and/or software used to wirelessly connect to the NovaMouse™ flexible cape.

USB Connection to PC And Software Installation To A Computer

The NovaMouse™ receiver base can be easily connected to a personal computer (PC) and/or other computing device via a universal serial bus (USB) and/or other data connection for data transfer. NovaMouse™ software can be installed on a computer, and the NovaMouse™ receiver base can be connected to the computer (with the NovaMouse™ software installed thereon) so that the computer can provide data analysis and/or display of the pulse and/or oxygen saturation (SaO₂) data.

Discussion Of The Figures

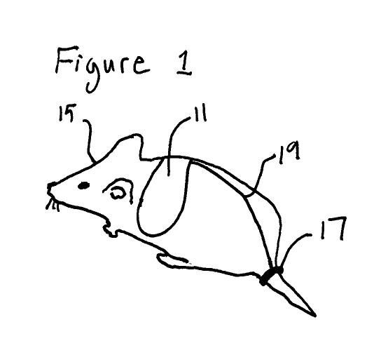

According to embodiments of the present invention illustrated in FIG. 1, the flexible cape 11 may be attached to the abdominal region of the mouse 15, for example, using an elastic strap and/or sutures, and the cuff 17 may be attached around the tail of the mouse 15. The cuff 17 may be a ring configured to fit around the tail, and the cape 11 and the cuff 17 may be electrically coupled using a wired coupling 19 configured to extend along the back of the mouse 15 between the cape 11 and the cuff 17.

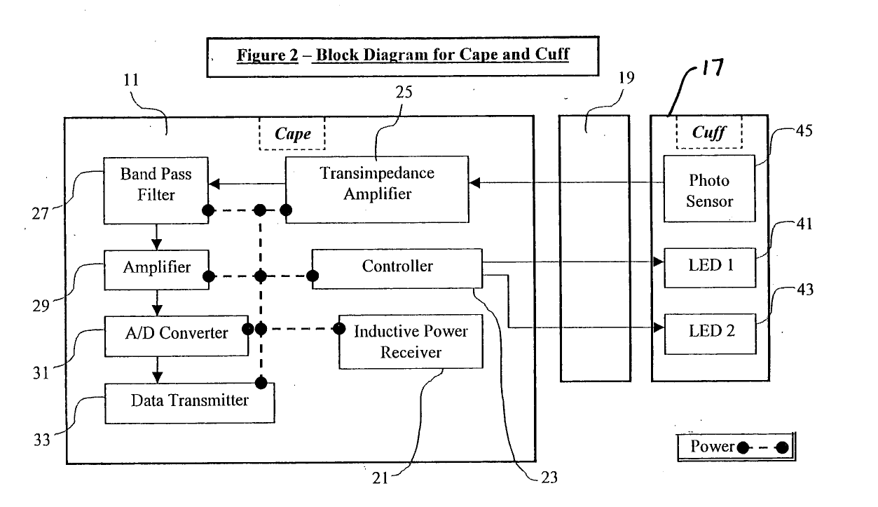

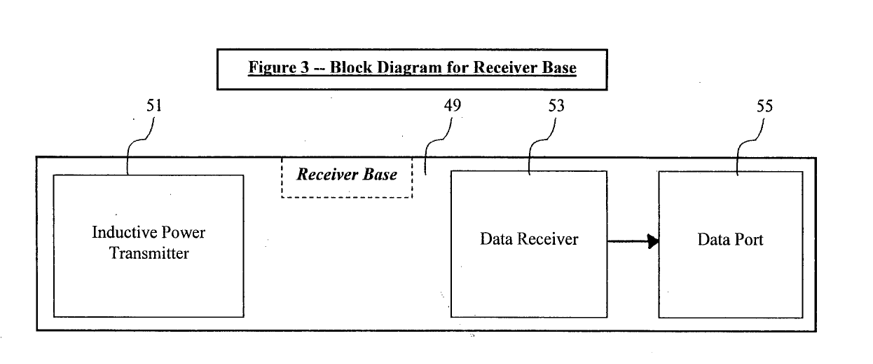

FIG. 2 is a block diagram illustrating pulse oximeters including a cape 11, a cuff 17, and a wired coupling 19 between the cape and the cuff according to embodiments of the present invention. Referring to FIG. 2, the cape 11 may include an inductive power receiver 21, a controller 23, a transimpedance amplifier 25, a filter 27, an amplifier 29, an analog-to-digital converter 31, and a data transmitter 33. The cuff 17 may include a first light emitting diode (LED) 41, a second light emitting diode (LED) 43, and a sensor 45. As shown, the wired coupling 19 may provide a wired coupling from the controller 23 to the first and second LEDs, and a wired coupling from the sensor 45 to the transmipednace amplifier 25. FIG. 3 is a block diagram illustrating a receiver base 49 for use with pulse oximeters illustrated in FIG. 2. Referring to FIG. 3, the receiver base may include an inductive power transmitter 51, a data receiver 53, and a dataport 55.

Referring to FIGS. 2 and 3, operation of a pulse oximeter according to embodiments of the present invention will now be discussed. The receiver of FIG. 3 may be placed under a cage housing a mouse wearing the cape 11 and the cuff 17. The inductive power transmitter 51 generates an electromagnetic field within the cage, and the inductive power receiver 21 generates electrical power used to power electrical components in the cape 11 and the cuff 17. Accordingly, a wired electrical coupling is not required to power the electrical components of the cape and cuff.

Responsive to receiving power from the inductive power receiver 21, the controller 23 generates electrical signals to power the first and second LEDs 41 and 43 in the cuff 17. More particularly, the controller 23 may alternatingly pulse the first and second LEDs 41 and 43. The first and second LEDs 41 and 43 may generate optical energy of different wavelengths. According to particular embodiments of the present invention, the first LED 41 may generate infrared optical energy, and the second LED 43 may generate optical energy having a wavelength of approximately 660 nm. At least a portion of the optical energy generated by each of the first and second LEDs 41 and 43 may be directed through the tail of the mouse and received by the sensor 45.

Responsive to receiving optical energy from one or both of the LEDs 41 and 43, the sensor 45 generates an electrical signal that is transmitted from the cuff 17 to the cape 11 via the wired coupling 19. The sensor 45 may include a photodiode that generates a current responsive to optical energy. The electrical signal from the sensor 45 may be amplified by the transimpedance amplifier 25, filtered by bandpass filter 27, amplified by amplifier 29, and converted to a digital signal using analog-to-digital converter 31. More particularly, the electrical signal from the sensor 45 may be a current signal, and the transimpedance amplifier 25 may convert the current signal to a voltage signal. The bandpass filter 27 may filter the voltage signal to frequencies within a range of approximately 0.1 Hz to 300 Hz. The filtered signal may be amplified by amplifier 29 so that the amplified signal fits within an operating range of the analog-to-digital converter 31. The digital signal generated by the analog-to-digital converter 31 can then be transmitted by the data transmitter 33 to the data receiver 53 of the receiver base without a wired coupling therebetween.

The data receiver 53 of the receiver base 49 may provide the received data through a data port 55 (such as a universal serial bus port) to a computing device such as a personal computer for analysis and/or display of pulse and/or oxygen saturation information. In an alternative, data analysis and/or display may be integrated in the receiver base 49.

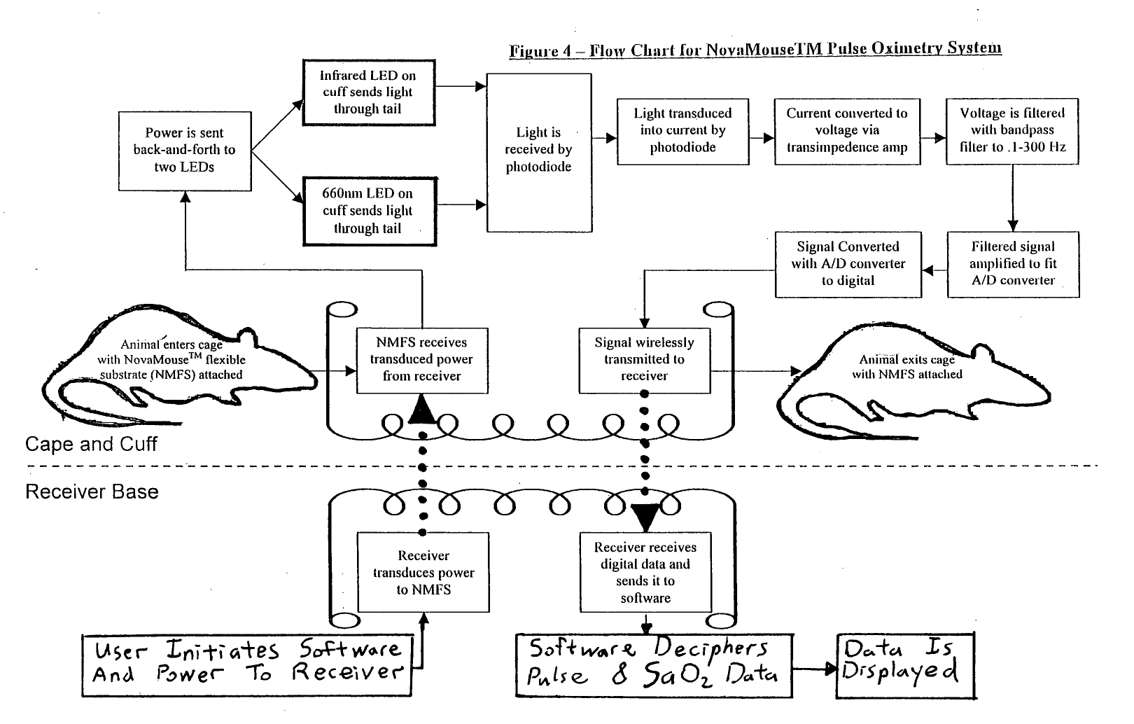

Moreover, the electrical and/or electronic components providing the functionality of the cape 11 may be packaged on a flexible substrate such as a polyimide substrate. Accordingly, the cape 11 may conform to the mouse's body. FIG. 4 is a system flow chart for pulse oximeters according to embodiments of the present invention.

The present invention has been described with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings and specification, there have been disclosed embodiments of the invention and, although specific terms are employed, they are used in a generic and descriptive sense only and not for purposes of limitation.

For example, aspects of the present invention may be embodied in monitoring devices other than pulse oximeters. Moreover, aspects of the present invention may be embodied in monitoring devices for laboratory animal subjects other than mice. According to embodiments of the present invention, a “subject” can be any animal subject, and may preferably be a mammalian subjects (e.g., canines, felines, bovines, caprines, ovines, equines, rodents, porcines, and/or lagomorphs). The term “small animal” includes mice, rats, guinea pigs, dogs, cats, monkeys, pigs, and rabbits. Embodiments of the present invention may be particularly suitable for use with small animals such as mice undergoing laboratory investigational studies.

In the drawings and specification, there have been disclosed typical preferred embodiments of the invention and, although specific terms are employed, they are used in a generic and descriptive sense only and not for purposes of limitation, the scope of the invention being set forth in the following claims. As used herein, the term “comprising” or “comprises” is open-ended, and includes one or more stated elements, steps, and/or functions. More particularly, it should be emphasized that the term “comprises/comprising” when used in this specification is taken to specify the presence of stated features, integers, steps or components but does not preclude the presence or addition of one or more other features, integers, steps, components or groups thereof.

1. A monitoring device for a laboratory animal, the monitoring device comprising:
an inductive power receiver configured to generate electrical power responsive to an electromagnetic field;
a sensor configured to generate an electrical signal responsive to an aspect of the laboratory animal being monitored; and
a data transmitter configured to wirelessly transmit data responsive to the electrical signal generated by the sensor and responsive to the electrical power generated by the inductive power receiver.

2. A monitoring device according to claim 1 further comprising:
at least one LED configured to transmit optical energy through a portion of the laboratory animal responsive to power generated by the inductive power receiver, wherein the sensor is further configured to receive a portion of the optical energy transmitted through the laboratory animal and to generate the electrical signal responsive to the received optical energy.

3. A monitoring device according to claim 1 wherein the laboratory animal comprises a small animal.

4. A monitoring device according to claim 1 wherein the laboratory animal comprises a mouse.
